(19) United States
(12) Patent Application Publication
(10) Pub. No.: US 2006/0100486 A1
(43) Pub. Date: May 11, 2006

(54) Endoscopic capsule for observing tumors

(75) Inventor: Michael Maschke, Lonnerstadt (DE)

Correspondence Address: SIEMENS CORPORATION;INTELLECTUAL PROPERTY DEPARTMENT, 170 WOOD AVENUE, SOUTH, ISELIN, NJ, 08830, US

(73) Assignees: Siemens; Aktiengesellschaft

(21) Appl. No.: 11/312,712

(22) Filed: Dec. 21, 2005

Related U.S. Application Data
(63) Continuation-in-part of application No. PCT/EP04/06259, filed on Jun. 9, 2004.

(30) Foreign Application Priority Data
Jun. 16, 2003 (DE) 10327034.5

(51) Int. Cl. A61B 1/06; A61B 6/00

(52) U.S. Cl. 600/160; 600/109; 600/476; 600/179

(57) ABSTRACT

An endoscopic capsule for wireless transmission of medical images is provided. The capsule comprises a light transmitting unit and a light receiver. If a diseased tissue contains a contrast medium concentrated in the diseased tissue and the diseased tissue is irradiated by excitation light emitted by the light transmitting unit at a specific frequency, fluorescent light detectable by the light receiver occurs at a frequency different from the specific frequency. The light transmitting unit is arranged at a leading end of the capsule and the light receiver is arranged at a trailing end of the capsule.

### CROSS REFERENCE TO RELATED APPLICATIONS

This application is a continuation-in-part application of the International Application No. PCT/EP2004/006259, filed Jun. 9, 2004 and claims the benefit thereof. This application further claims the benefit of German Application No. 10327034.5, filed Jun. 16, 2003 the priority of which has been claimed by the above-mentioned International Application No. PCT/EP2004/006259. Each of the above-referenced applications are incorporated by reference herein by their entirety.

### FIELD OF INVENTION

The invention relates to an endoscopic capsule having a light transmitting unit and a light receiver for recording images transmittable wirelessly to an external receiver from within an organ or vessel, in particular from the gastrointestinal tract in the body of a human being or animal, with said light transmitting unit emitting light at one frequency, which light will result in the radiation of fluorescent light at a different frequency when a contrast medium concentrates in the diseased tissue.

### BACKGROUND OF INVENTION

DE 101 09 539 A1 describes a device that enables cancers to be selectively examined and depicted because the fluorescing agent will selectively concentrate in the tumor cells and hence enable good contrast imaging when irradiated with excitation light. The known device being suitable only for external body examinations, it can only identify cancers sited relatively close to the skin.

Cancers of the esophagus and digestive (gastrointestinal) tract are of a type most frequently having a fatal outcome. Early detection, diagnosis, and therapy are extremely important for a favorable prognosis.

WO 02/058531 A2 discloses a swallowable capsule that can be provided with various image recording systems. The recorded images are transmitted wirelessly to outside the body. A capsule for recording fluorescence is provided therein at whose leading end are located a light transmitter and a light receiver, with a filter being required for separating the different types of light.

WO 02/036007 A1 discloses a swallowable capsule that includes LEDs for exciting a fluoroscopic substance previously taken by the examinee, a filter, and an image receiver at one end of the capsule.

### SUMMARY OF INVENTION

An object of the invention is to provide an endoscopic capsule enabling early detection and diagnosis of cancers inside the body of a human being or animal, in particular in the gastrointestinal tract.

To achieve said object, it is inventively provided in an endoscopic capsule of the type mentioned at the beginning for the light transmitter to be located at the leading end and the light receiver to be located at the trailing end of the endoscopic capsule.

Said variant is readily implementable because while passing through the gastrointestinal tract or a vessel the endoscopic capsule will irradiate the tissue, and hence also specifically tumorous tissue containing concentrated contrast medium, by means of the light transmitting unit at the front end, with the fluorescent radiation of said tumorous tissue naturally persisting after the capsule has moved past so that it can record said fluorescent radiation using the light receiver located at its back end then convert it via an image processing unit into an optical image.

The embodiment is preferably such that, alongside a drive unit for the excitation light of the light transmitting unit as well as the light receiver, a power supply unit, an image preprocessing unit, and a transmitting and receiving unit are located in the endoscopic capsule, with said light transmitting unit being able to include preferably a laser diode and the light receiver being able to include to particularly practical effect a CCD camera.

The solution according to the invention eliminates the known disadvantage of all previously known fluorescing optical imaging solutions, namely their shallow depth of tissue penetration.

The scope of the invention finally also includes providing the endoscopic capsule with a magnet enabling it to be moved by means of a magnetic field applied outside the body.

### BRIEF DESCRIPTION OF THE DRAWINGS

Further advantages and specifics of the invention will emerge from the following description of an exemplary embodiment with reference to the drawings, in which:

FIG. 1 is a schematic of an inventive endoscopic capsule inside a vessel or the gastrointestinal tract with the tissue being excited by the light transmitting unit, and

FIG. 2 is a schematic of the endoscopic capsule having at a later instant been moved on showing the fluorescent light being recorded from the tumorous tissue.

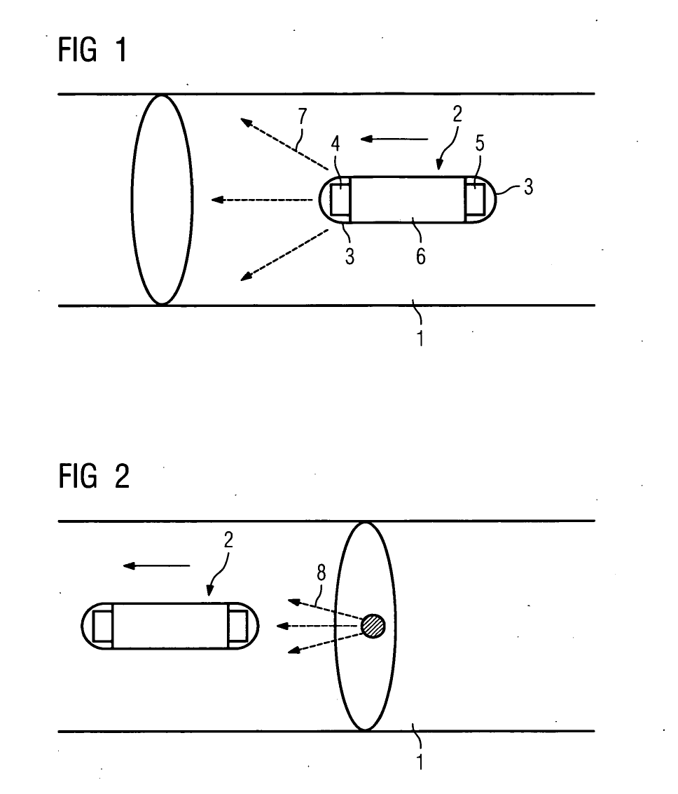

### DETAILED DESCRIPTION OF INVENTION

Inside a channel 1 in the body of a human being or animal, which is to say, for example, inside a vessel or the gastrointestinal tract, is an endoscopic capsule 2 provided at its front end having a transparent cover 3 with a light transmitting unit 4 and at its back end 5, likewise under a transparent cover 3, with a fluorescent light receiver 5. The central section 6 contains the power supply unit, the transmitting and receiving unit, the image preprocessing unit, and a drive unit for the excitation light of the light transmitting unit 4 preferably containing a laser diode. The fluorescent light receiver 5 preferably contains a CCD camera.

The tissue of the gastrointestinal tract or vessel is irradiated by the excitation light 7 of the light transmitting unit 4 and the concentrated contrast medium in any tumorous tissue present will be excited thereby and emit a fluorescent radiation 8 which, after the endoscopic capsule has accordingly moved on, can be recorded by means of the receiving unit at its back end, evaluated, then radiated to an external location, where it can be shown on a display for the physician, via the transmitting and receiving unit.

The invention is not restricted to the exemplary embodiment illustrated. The endoscopic capsule could also be provided with a magnet enabling the capsule to be moved within the body under the control of a magnetic field applied from outside the body.

1. An endoscopic capsule for wireless transmission of medical images to an external receiver, comprising:
a leading end;
a trailing end;
a light transmitting unit arranged at the leading end; and
a light receiver arranged at the trailing end for recording medical images from within an organ or vessel of a body, wherein
the light transmitting unit is configured to emit excitation light at a specific frequency to a diseased tissue, the excitation light triggering radiation of fluorescent light having a frequency different from the specific frequency, the radiation of fluorescent light occurring when a contrast medium is concentrated in the diseased tissue and the diseased tissue is irradiated by the excitation light.

2. The endoscopic capsule as claimed in claim 1, wherein the medical images are recorded in an gastrointestinal tract within the body of a human being or an animal.

3. The endoscopic capsule as claimed in claim 1, further comprising:
a driver connected to the light transmitting unit for generating the emitted light;
a power supply unit;
an image processing unit; and
a transmitter-receiver unit.

4. The endoscopic capsule as claimed in claim 1, wherein the light receiver includes a CCD camera.

5. The endoscopic capsule as claimed in claim 1, wherein the light transmitting unit includes a laser diode.

6. The endoscopic capsule as claimed in claim 1, further comprising a magnet for magnetically navigating the endoscopic capsule by an external magnetic field applied to the magnet from outside the body.
